SEQUENCE LISTING (3 sequences)

SEQ ID NO: 1 - DNA, 24 bp, ARTIFICIAL
  forward PSMA primer
tgagagactccaggactttgacaa

SEQ ID NO: 2 - DNA, 25 bp, ARTIFICIAL
  reverse PSMA primer
ggatcaataaatgctctttccagaa

SEQ ID NO: 3 - DNA, 40 bp, ARTIFICIAL
  PSMA probe
agcaacccaatagtattaagaatgatgaatgatcaactca

(19) United States
(12) Patent Application Publication
(10) Pub. No.: US 2006/0140871 A1
(43) Pub. Date: Jun. 29, 2006

(54) Magnetic resonance imaging of prostate cancer

(76) Inventor: Laurel O. Sillerud, Albuquerque, NM (US)

Correspondence Address: MUETING, RAASCH & GEBHARDT, P.A., P.O. BOX 581415, MINNEAPOLIS, MN, 55458, US

(21) Appl. No.: 11/290,765

(22) Filed: Nov. 30, 2005

Related U.S. Application Data
(60) Provisional application No. 60/631,725, filed on Nov. 30, 2004.

(51) Int. Cl. A61K 49/16

(52) U.S. Cl. 424/9.36; 977/930

(57) ABSTRACT

Paramagnetic or superparamagnetic nanoparticle-ligand conjugates that include a recognition ligand that interacts with a component on the surface of a prostate cancer cell. Nanoparticle-ligand conjugates of the invention may be used for magnetic resonance imaging of prostate cancer, or for treatment of tumors by targeted thermal ablation.

This application claims the benefit of U.S. Provisional Application Ser. No. 60/631,725, filed Nov. 30, 2004, which is incorporated by reference herein.

### BACKGROUND

Magnetic resonance imaging (MRI) is widely used for obtaining spatial images of human subjects for clinical diagnosis. Advantages of using this procedure over other diagnostic methods such as x-ray computer-aided tomography (CT), are generally recognized. For instance, the magnetic fields utilized in an MRI scan do not appear to have any ill effects on human health. In addition, while x-ray CT images are formed from the observation of a single parameter, i.e., x-ray attenuation, magnetic resonance images are a composite of the effects of a number of parameters that are analyzed and combined by computer, providing richer and more comprehensive analysis. Choice of the appropriate instrument parameters such as radio frequency (Rf), pulsing and timing can also be utilized to enhance or attenuate the signals of particular image-producing parameters, thereby improving the image quality. MRI has also proven to be a valuable diagnostic tool for distinguishing normal and diseased tissue, as these tissues possess different parameter values that can be differentiated in the image prepared.

To obtain an image of an organ or tissue using MRI, a subject is placed in a strong external magnetic field and the effect of this field on the magnetic properties of the protons (hydrogen nuclei) contained in and surrounding the organ or tissue is observed. The proton relaxation times, termed T₁ and T₂, are of primary importance. T₁ (also called the spin-lattice or longitudinal relaxation time) and T₂ (also called the spin-spin or transverse relaxation time) depend on the chemical and physical environment of organ or tissue protons and are measured using the Rf pulsing technique. This information is then analyzed as a function of distance by a computer, which uses it to generate an image.

Unfortunately, the image produced often lacks definition and clarity due to the similarity of the signal from other tissues. To generate an image with good definition, the T₁ and/or T₂ of the tissue to be imaged must be distinct from that of the background tissue. One approach to increase these differences is to use contrast agents. MRI contrast agents either act predominantly on T1 relaxation, which results in signal enhancement and “positive” contrast, or on T2 relaxation, which results in signal reduction and “negative” contrast. The T1 and T2 values are changed by changing the number of fluctuating magnetic fields near a nucleus. A variety MRI contrast agents are available that can be categorized by their magnetic properties.

Paramagnetic materials may been used as MRI contrast agents because of their ability to decrease T₁ (Weinmann et al., Am. J. Rad. 142, 619 (1984)). Paramagnetic materials are characterized by a weak, positive magnetic susceptibility and by their inability to remain magnetic in the absence of an applied magnetic filed. Ferromagnetic materials have also been used as contrast agents because of their ability to decrease T₂ (Olsson et al., Mag Res. Imaging 4, 437 (1986)). Ferromagnetic materials have high, positive magnetic susceptibilities and maintain their magnetism in the absence of an applied field. A third class of magnetic materials, termed superparamagnetic materials, have also been used as contrast agents (Saini et al., Radiology, 167, 211 (1987)). Like paramagnetic materials, superparamagnetic materials are characterized by an inability to remain magnetic in the absence of an applied magnetic field. Superparamagnetic materials can have magnetic susceptibilities nearly as high as ferromagnetic materials and far higher than paramagnetic materials (Bean and Livingston J. Appl. Phys. Suppl to vol. 30, 1205 (1959)).

Prostate cancer, a carcinoma, is the most common cancer other than superficial skin cancer, and is the second leading cause of cancer death in American men. Furthermore, between 1976 and 1994, prostate cancer rates doubled and mortality increased by 20% (Haas G. & Sakr W., CA Cancer J. Clin., 47, 273-287 (1997)). The reasons for the increase are not known, but increasing life expectancy, growing disease prevalence resulting from environmental carcinogens, and increasing use of novel diagnostic modalities have been suggested as causes. Most prostate cancers are slowly progressive malignancies, and many are present for years before they are identified by clinical diagnosis. In the early stages, the disease stays in the prostate and is not life threatening, but without treatment it metastasizes to other parts of the body and eventually causes death. Early detection, and evaluation of the course of the disease, are crucial for determining the appropriate therapeutic regimen.

Current prostate cancer screening using serum prostate specific antigen (PSA) testing has resulted in a large increase in the detection rate for prostate cancer, but PSA levels often rise due to prostate pathology independent of adenocarcinoma. Lieberman, Am J Ther. 2004; 11:501-6; Fitzpatrick, BJU Int. 2004 March; 93 Suppl 1:2-4. PSA is also a soluble marker disseminated into the circulation, rather than a cell-surface marker that could be used to image primary tumors or distant metastases.

One cell surface antigen overexpressed in tumors is prostate specific membrane antigen (PSMA), a transmembrane glycoprotein highly expressed by most prostate cancers. (Silver et al., Clin Cancer Res. 1997; 3:81-85). PSMA is also expressed on the tumor vascular endothelium of virtually all solid carcinomas and sarcomas but not on normal vascular endothelium (Chang et al., Clin. Cancer Res. 1999; 5(10):2674-81). PSMA expression correlates with tumor grade, pathological stage, aneuploidy, and biochemical recurrence (Ross et al., Clin Cancer Res 2003 Dec. 15; 9(17):6357-62).

Antibodies have been used to target PSMA. One antibody, 7E11-C5 (capromab), binds to the intracellular domain, which only becomes accessible upon cell death. (Troyer et al., Prostate 1997 Mar. 1; 30(4):232-42). More recently, antibodies that bind to the extracellular domain of PSMA have been developed. For example, in clinical trials, ¹³¹I- and ¹¹¹In-labeled monoclonal antibodies J415, J533 and J591 have successfully located LNCaP metastases in nude mice (Smith-Jones et al., J Nucl Med 2003; 44:610-617). Additionally, several phase I studies using radiolabeled or cytotoxin (DM1) linked J591 have demonstrated excellent tumor targeting. See Bander et al., J Clin Oncol. 2005 Apr. 18; [Epub ahead of print]; Milowsky et al., J Clin Oncol. 2004 Jul. 1; 22(13):2522-31; Nanus et al., J Urol. 2003; 170(6 Pt 2):S84-8; discussion S88-9; and Bander et al., J Urol 2003; 170(5): 1717-21. Monoclonal antibody (MAb) 3C6 has also been shown to target the extracellular domain of PSMA. (Tino et al., Hybridoma. 2000 June; 19(3):249-57).

An imaging technique that could preferentially target cancer cells would constitute a welcome advance in the art of prostate cancer detection.

### SUMMARY OF THE INVENTION

The present invention provides, in one aspect, a nanoparticle-ligand conjugate that includes at least one paramagnetic or superparamagnetic nanoparticle; and at least one recognition ligand that selectively binds to a component on the surface of a prostate cancer cell. In one embodiment, the nanoparticle of the nanoparticle-ligand conjugate includes iron oxide.

In an additional embodiment of the nanoparticle-ligand conjugate of the invention, the nanoparticle-ligand conjugate includes a plurality of recognition ligands. In a further embodiment, the plurality of recognition ligands includes a plurality of different recognition ligands.

In a further embodiment of the nanoparticle-ligand conjugate of the invention, the nanoparticle-ligand conjugate includes a plurality of nanoparticles. Embodiments including a plurality of nanoparticles may further include a plurality of recognition ligands. In further embodiments, these recognition ligands may include a plurality of different recognition ligands.

In yet another embodiment of the nanoparticle-ligand conjugate of the invention, the nanoparticle-ligand conjugate includes a recognition ligand that binds to prostate surface membrane antigen (PSMA). In another embodiment of the nanoparticle-ligand conjugate, the recognition ligand may be an antibody. Embodiments of the invention including antibodies may further include monoclonal antibodies.

In a further embodiment of the nanoparticle-ligand conjugate of the invention, the nanoparticle of the nanoparticle-ligand conjugate includes a superparamagnetic particle.

In another aspect, the present invention provides a method for diagnosing prostate cancer in a subject that includes contacting prostate tissue of a subject with the nanoparticle-ligand conjugate that includes at least one paramagnetic or superparamagnetic nanoparticle and at least one recognition ligand that selectively binds to a component on the surface of a prostate cancer cell; applying a magnetic field to the prostate tissue; and detecting nanoparticle-ligand conjugate bound to the prostate tissue. The presence of conjugate bound to the prostate tissue as a result of this method is indicative of prostate cancer in the subject.

In an embodiment of the method for diagnosing prostate cancer, the recognition ligand binds to prostate surface membrane antigen (PSMA). In another embodiment, the recognition ligand includes an antibody.

In further embodiments of the method for diagnosing prostate cancer, the step of contacting prostate tissue of the subject with the nanoparticle-ligand conjugate is performed in vivo. In another embodiment, the step of contacting prostate tissue of the subject with the nanoparticle-ligand conjugate is performed in vitro.

In additional embodiments of the method, the nanoparticle-ligand conjugate is detected using molecular resonance imaging (MRI). Further embodiments of the invention are directed to the detection of metastatic prostate cancer tissue.

A further aspect of the invention provides a method for treating prostate cancer in a subject that includes contacting prostate tumor tissue of the subject with the nanoparticle-ligand conjugate of claim 1 such that the conjugate binds to the tumor tissue; and irradiating the tumor tissue to result in thermal ablation of the tumor tissue.

Embodiments of the method for treating prostate cancer may include a recognition ligand that binds to prostate surface membrane antigen (PSMA). In further embodiments of the method for treating prostate cancer, the recognition ligand includes an antibody. The method for treating prostate cancer may also be used, in additional embodiments, to treat metastatic prostate cancer.

Unless otherwise specified, “a,” “an,” “the,” and “at least one” are used interchangeably and mean one or more than one.

### BRIEF DESCRIPTION OF THE FIGURES

FIGS. 1A-1B show (1A) Real-time RT-PCR analysis of PSMA expression in PrEC, LNCaP, C4-2, DU-145, PC-3, tumor and normal prostate tissue. Values are normalized for RNA input using GAPDH; relative expression is compared to LNCaP (=100%); numbers above bars indicate actual values in percent of LNCaP; error bars represent duplicate values±standard deviations. (1B) Flow cytometric analysis of the expression of PSMA by several types of cultured human prostate cells. The cells marked normal and cancer were derived from a culture developed from a prostatectomy sample.

FIGS. 2A-2D show MR Imaging of human prostate cancer cells labeled with an antibody to PSMA conjugated to superparamagnetic nanoparticles referred to herein by the trade name DYNABEADS. Two types of cells were used, LNCap cells, which display PSMA, and DU-145 cells, which do not display PSMA. (2A) Spin-echo MR image, TE=15.5 milliseconds (ms). (2B) Gradient-echo MR image, TE=4 ms. (2C) Imaginary component of FID projection image, TE=0. (2D) Photograph of the NMR sample used. Note the air bubble in the sample, which appears with different sizes in A and B due to field gradient effects.

FIGS. 3A-3C show measured contrast values for the (3A) Spin-echo image, (3B) the Gradient-echo image, and (3C) the magnitude, imaginary and phase images of the NMR sample from FID projection data. The errors are derived from the standard deviations of the pixel values in regions of interest surrounding the DU-145 and LNCaP cells.

FIGS. 4A-4C show MR Images of the MACS microbeads sample. MACS microbeads are referred to herein by the trade name MACS. (4A) T₁-weighted spin-echo image, TR=0.5 ms. (4B) Spin-echo image, TR=12 s. T₁-weighting has been eliminated using the long T_{R}, but weak T₂ contrast is evident as hypointensity of the LNCaP cell band. (4C) T₁-weighted FID projection image (magnitude).

FIGS. 5A-5B show (5A) Contrast as a function of repetition time (TR) for T₁-weighted SE images of the MACS sample. (5B) Contrast in the FID projection image of the MACS sample.

FIG. 6 shows the magnetic field (solid black lines) due to a superparamagnetic nanoparticle. The field lines indicate the direction of the magnetic field in the plane of the paper. The dotted line indicates a possible path taken by a water molecule that diffuses from a position where the magnetic field from the particle points up to a position where the field points down. In this case, the displacement of the water molecule, indicated by the arrow, is approximately equal to d, the diameter of the nanoparticle.

FIGS. 7A-7C show imaging of a large subcutaneous LNCaP tumor in a mouse. In (7A), a control test tube containing LNCaP cells and DE-145 cells shows brightening of the cancerous LNCaP cells. In (7B), the tumor intensity after 30 minutes is shown, while in (7C) the tumor intensity after 23 hours is shown. The tumor is distinctly more visible at 23 hours.

FIG. 8 is a bar graph showing that tumor image intensity relative to control increased by a factor of two.

### DETAILED DESCRIPTION OF ILLUSTRATIVE EMBODIMENTS OF THE INVENTION

The present invention is directed to paramagnetic and superparamagnetic nanoparticle-ligand conjugates and their use to detect and/or treat prostate cancer, both primary and metastatic, as well as precancerous conditions. Embodiments of the invention include nanoparticles conjugated to recognition ligands that interact with prostate cancer cell surface components such as, for example, prostate specific membrane antigen (PSMA). Binding of the paramagnetic or superparamagnetic nanoparticle-ligand conjugates to prostate cancer cells may be detected, for example, by magnetic resonance imaging. Prostate cancers can be detected locally, in the prostate; and the invention allows metastatic disease can be detected at remote locations as well. Conjugation of ligands that recognize prostate cancer cell surface components to paramagnetic or superparamagnetic nanoparticles offers increased specificity and sensitivity.

Paramagnetic and Superparamagnetic Nanoparticles

Paramagnetic and superparamagnetic nanoparticles are nanoparticles that exhibit a dipole moment in the presence of a magnetic field, but lose it when the magnetic field is removed. Paramagnetic materials have a relatively small positive magnetic susceptibility (magnetability), while superparamagnetic materials have a much larger magnetic vector or magnetic moment.

Paramagnetic nanoparticles preferably contain transition metal ions, such as iron, manganese, gadolinium, and/or copper ions. Additional materials suitable for preparation of paramagnetic particles include transition metals such as titanium, vanadium, chromium, cobalt, and nickel, lanthanide metals such as europium, and/or actinide metals such as protactinium. These metals may be independently selected or excluded for use in different embodiments of the invention. Paramagnetic ions have unpaired electrons, resulting in a positive magnetic susceptibility. Preferably, the paramagnetic nanoparticles contain a relatively non-toxic metal such as iron.

Superparamagnetic nanoparticles are composed of substances like ferrite which are ferromagnetic in bulk but which, because of the very small particle size, have lost their permanent magnetism. Generally, superparamagnetic particles have a particle size that ranges from about 30 to 50 nanometers (about 300 to 500 angstroms (Å)). Particles in this size range are impacted by both thermal effects, which quench the magnetic field, and magnetic ordering effects, with the result that the magnetic vector is unstable and fluctuates in the same way as for paramagnetic materials. Superparamagnetic materials possess high magnetic susceptibility and crystalline structures found in ferromagnetic materials, but rapidly lose their magnetic properties in the absence of an applied magnetic field. Superparamagnetic materials are preferably iron oxides such as magnetite (Fe₃O₄) or γ-ferric oxide (Fe₂O₃). Superparamagnetic particles exhibit stronger magnetic effects than paramagnetic particles of an equivalent size. For example, an iron oxide superparamagnetic particle may exhibit a magnetic field that is about 50,000 times stronger than the magnetic field exhibited by a similarly-sized gadolinium-based paramagnetic particle.

Preferably, the nanoparticle used in the invention is a superparamagnetic iron oxide nanoparticle, also known as a “SPION.” Superparamagnetic iron oxide (SPIO) particles can be manufactured with different particle sizes and surface coatings. Large SPIO particles (about 50-150 nm) predominantly produce a signal decrease or T2-shortening and have been used as contrast media for MRI of the liver and spleen. They have a high accuracy, and some have been shown to be especially effective in detecting liver metastases (approved for clinical use: AMI-25 (ferumoxide, available under the tradename ENDOREM or FERIDEX), and SHU-555A (ferucarbotran, available under the tradename RESOVIST)). Smaller particles (about 20 nm in diameter), also known as “ultrasmall” SPIO (USPIO), show a different organ distribution and have a potential for improving noninvasive lymph node assessment or characterizing vulnerable atherosclerotic plaques (in clinical trials: AMI-227 (ferumoxtran, available under the tradenames SINEREM or COMBIDEX). FERIDEX and COMBIDEX are available from Advanced Magnetics, Inc., Cambridge Mass.

Superparamagnetic iron oxide nanoparticles (SPIONs) offer significant advantages over other potential contrast agents in that they have very large effects, which are propagated over long distances, as a result of their extremely large induced magnetic moments. The surrounding water molecules, in the case of MRI, act as signal amplifiers and detectors for the magnetic field gradients induced by the nanoparticles. SPIONS also exhibit improved pharmacokinetics relative to other contrast agents. Injected intravenously, SPIONs slowly extravasate from the vascular into the interstitial space, where they travel through the intersitital-lymphatic fluid to target small nodal metastases (Harisinghani et al., N Engl J Med 2003 Jun. 19; 348(25):2491-9. Erratum in: N Engl J. Med. 2003 Sep. 4; 49(10):1010).

Prostate Cell Surface Components

The paramagnetic or superparamagnetic nanoparticle-ligand conjugates of the invention are directed by the recognition ligand to a targeted cell surface component of prostate tumor cells. The cell surface component is preferably a biomolecule that is specifically or preferentially associated with cancer, particularly prostate cancer. The cell surface, as defined herein, is the outer surface of the cell that is accessible to interaction with a recognition ligand. An example of a cell surface component that can be targeted is a transmembrane receptor. While cell surface polypeptides are generally preferred targets, other cell surface materials capable of selective or specific binding to a ligand, such as oligosaccharides and lipids, may also be used as targets for the recognition ligands of nanoparticle-ligand conjugates.

Examples of cell surface components that can be targeted in accordance with the invention include cell surface components associated with prostate cancer cells such as prostate-specific membrane antigen (PSMA), prostate stem cell antigen (PSCA), carcinoembryonic antigen (CEA) and a biomolecule known as six-transmembrane epithelial antigen of the prostate (STEAP). PSMA is expressed in virtually 100% of all prostate cancers, so it represents a particularly attractive target for the diagnostic and therapeutic methods of the invention. The crystal structure of PSMA has been determined and can be used to facilitate the preparation of recognition ligands that selectively bind to PSMA. See Davis et al., Proc Natl Acad Sci USA. 2005 Apr. 26; 102(17):5981-6.

Recognition Ligands

The paramagnetic or superparamagnetic nanoparticle-ligand conjugate includes a recognition ligand that selectively binds to a component on the surface of a prostate cancer cell. As used herein, the phrase “selectively binds” and other permutations of the phrase refer to a recognition ligand (e.g., an antibody) that will, under appropriate (e.g., physiological) conditions, interact with an a cell surface component (e.g., an antigen) preferentially compared to a different or structurally unrelated cell surface component. Recognition ligands include antibodies and other types of proteins, peptides, small organic molecules, and the like that selectively bind to a component of the surface of a tumor cell. For example, neurotensin can serve as a suitable non-antibody recognition ligand.

The paramagnetic or superparamagnetic nanoparticle may be conjugated to a single recognition ligand. Alternatively, the paramagnetic or superparamagnetic nanoparticle may be conjugated to a plurality of recognition ligands that target prostate cancer cells. The plurality of recognition ligands can be the same type of ligand (e.g., all antibodies) or they may include differing types of recognition ligands. Further, when the superparamagnetic nanoparticle is conjugated to a plurality of recognition ligands, the ligands may selectively bind to a single cell surface component, or they may bind to a variety of different cell surface components.

In other embodiments, sensitivity is increased, or the signal is amplified, by conjugating multiple paramagnetic or superparamagnetic nanoparticles to a single recognition ligand. Alternatively, several paramagnetic or superparamagnetic nanoparticles can be conjugated to a linker that is then linked to the one or more recognition ligands, such as an antibody.

In a preferred embodiment, the recognition ligand is an antibody that selectively binds the targeted cell surface component on the prostate cancer cell. For example, the antibody may selectively bind to prostate specific membrane antigen (PSMA), prostate stem cell antigen (PSCA), carcinoembryonic antigen (CEA) and a biomolecule known as six-transmembrane epithelial antigen of the prostate (STEAP). In a particularly preferred embodiment, the invention is directed to a conjugate that comprises a paramagnetic or superparamagnetic nanoparticle and an anti-PSMA antibody. In some embodiments, the cell surface component preferentially expressed by prostate cancer cells may be found in lower amounts in other prostate tissue. Preferably, the cell surface component used for ligand targeting is found at levels at least twice that found in non-cancerous prostate tissue, and more preferably at levels at least five times that found in non-cancerous prostate tissue. In further embodiments, the cell surface component is expressed exclusively by prostate cancer cells.

Antibodies, as defined herein, include vertebrate antibodies, hybrid antibodies, chimeric antibodies, humanized antibodies, altered antibodies, univalent antibodies, monoclonal and polyclonal antibodies, Fab proteins and single domain antibodies. Preferred types of antibodies used as recognition ligands for the present invention include monoclonal and polyclonal antibodies. These types of antibodies are generally prepared by differing procedures.

If polyclonal antibodies are desired, a selected animal (e.g., mouse, rabbit, goat, horse or bird, such as chicken) is immunized with the desired surface cell component of a prostate cancer cell. Serum from the immunized animal is collected and treated according to known procedures. Serum containing polyclonal antibodies to a prostate cancer cell surface component can be purified by immunoaffinity chromatography. Techniques for producing and processing polyclonal antisera are known in the art (see for example, Mayer and Walker eds. Immunochemical Methods in Cell and Molecular Biology (Academic Press, London) (1987), Coligan, et al., Unit 9, Current Protocols in Immunology, Wiley Interscience (1991), Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992)).

Monoclonal antibodies directed against a prostate cancer cell surface component are readily produced by one skilled in the art. The general methodology for making monoclonal antibodies by hybridomas is well known. Immortal antibody-producing cell lines can be created by cell fusion, and also by other techniques such as direct transformation of B lymphocyte cells with oncogenic DNA, or transfection with Epstein-Barr virus (See Monoclonal Antibody Production. Committee on Methods of Producing Monoclonal Antibodies, Institute for Laboratory Animal Research, National Research Council; The National Academies Press; (1999), Kohler & Milstein, Nature, 256:495 (1975); Coligan et al., sections 2.5.1-2.6.7; and Harlow et al., Antibodies: A Laboratory Manual, page 726 (Cold Spring Harbor Pub. 1988)). Panels of monoclonal antibodies produced against prostate cancer cell surface components can be screened for various properties such as epitope affinity.

Nanoparticle-Ligand Coupling

Nanoparticles are used as part of a nanoparticle-ligand conjugate and preferably include a ligand that recognizes a component on the surface of a prostate cancer cell. Nanoparticles may be conjugated to recognition ligands, also referred to herein as targeting agents, in various different ways. The coupling between the nanoparticle and the ligand can be covalent or non-covalent. Nanoparticles may be directly bound to a recognition ligand through reaction with an element of the nanoparticle. For example, nanoparticles containing iron oxide can be derivatized to ligands by linkage through the oxygen molecules of the iron oxide. In one embodiment, direct conjugation of iron oxide nanoparticles to a ligand (e.g., a monoclonal antibody) can be carried out through Schiff base formation and subsequent reduction. See Remsen et al., Am. J. Neuroradiol 17:411-418 (1996).

Alternatively, a paramagnetic or superparamagnetic nanoparticle may be chemically modified by the attachment of a functional group that in turn permits coupling to a ligand. For example, nanoparticles may be coupled to ligands using dimethyl suberimidate or glutaraldehyde. Other coupling agents include dicyclohexyl carbodiimide and n-hydroxy succinamide. Nanoparticles may also be associated with chelating agents that bind to the metals within the nanoparticle, and the chelating agents used as a coupling site for the ligand. In one embodiment, the chelating agent is a chelating protein. Examples of bifunctional chelating agents include diethylenetriamine-pentaacetic acid (DTPCA), imino-diacetic acid (IDA), nitrilo-triacetic acid (NTA), ethylenediamine-tetraacetic acid (EDTA), diaminocyclohexame-tetraacetic acid (DCTA), porphyrin, deferoxamine, and tetraagacyclo-tetradecane-tetraacetate (TETA).

The invention is not limited by the conjugation chemistry used to covalently link the nanoparticle to the recognition ligand. For example, conjugation may be carried out by attaching molecules with a high affinity for one another to the ligand and the nanoparticle. A preferred coupling chemistry involves avidin/biotin interactions. Avidin is a tetrameric glycoprotein present in egg whites that has an extremely high affinity for biotin, and binds to four molecules of biotin. The avidin-biotin interaction is an extremely strong non-covalent biological interaction between protein and a ligand. In particular embodiments of the invention, the paramagnetic or superparamagnetic nanoparticle can be derivatized with avidin or streptavidin, and the recognition ligand can be biotinylated, or vice versa. Combining the avidin-containing nanoparticle with the biotinylated recognition ligand under conditions that promote avidin/biotin binding results in the formation of a nanoparticle-ligand conjugate suitable for use in the method of the invention.

Alternatively or additionally, nanoparticles may be prepared for conjugation to a ligand by including them in particles that include a material (referred to herein as a complexing compound) that facilitates derivatization, such as polyethylene glycol, dextran or polyvinyl alcohol. For example, DYNABEADS are formed from superparamagnetic iron oxide particles suspended in a polystyrene matrix. A dextran coating on the iron oxide core prolongs their lifetime in the circulation, an advantage over gadolinium chelates, which generally undergo rapid renal elimination (Kircher et al., Cancer Res 2003 Dec. 1; 63(23):8122-5), and may prevent exposure to nanoparticles formed from toxic metals. Preferably, the complexing compound is coated over the one or more nanoparticles. A variety of oligo- and polysaccharides, protective colloids, and other compounds suitable for preparing complexed nanoparticles are described in U.S. Pat. No. 5,688,490 issued to Tournier et al. on Nov. 18, 1997. Preferably the complexing compound is one that prevents undesirable coalescence of particles that may lead to a loss of superparamagnetism, such as water-insoluble hydrophilic water-swelling substrates that tend to form gels in water. Coated nanoparticles generally have a size from about 70 to about 1000 nanometer radius, with particles having a size of about 200 nanometers radius being preferred. Particles that are too small may extravasate, while particles that are too large may be recognized and eliminated by immune cells such as macrophages.

Interaction of Nanoparticle-Ligand Conjugates with Prostate Cell Surface Components

Preferably, the recognition ligand selectively binds to a cell surface component of prostate cancer cells, but does not significantly bind to the cell surface components of other cells. Binding of the recognition ligand of the nanoparticle-ligand conjugate to a prostate cancer cell component brings the nanoparticle into proximity with the prostate cancer cell.

Detection of Prostate Cancer Cells Using Nanoparticle-Ligand Conjugates

Prostate cancer is cancer occurring in the prostate gland. The prostate gland is a gland in the male reproductive system that makes and stores a component of semen, and is located near the bladder. The stages generally used to categorize prostate cancer include the localized stage, in which the tumor is nonpalpable or is palpable but confined to the prostate gland; the regional stage, in which the tumor has grown through the prostate capsule and into, for instance, seminal vesicles or nearby muscles and organs; and the metastatic stage, which includes tumors that have metastasized to the pelvic lymph nodes, more distant parts of the body, or the combination thereof. The metastatic stage is also referred to herein as metastatic prostate cancer. The term “prostate cancer,” as used herein, refers to all three of these stages of cancer. The nanoparticle-ligand conjugates of the invention can also be used to detect or treat precancerous conditions, including enlarged prostate and prostatic intraepithelial neoplasia (PIN). The nanoparticle-ligand conjugates may also be useful for distinguishing precancerous conditions from benign prostate hyperplasia (BPH), a non-cancerous condition.

The present invention provides a method for diagnosing prostate cancer or a precancerous condition in a subject that includes contacting tissue of the subject, preferably prostate tissue, with the paramagnetic or superparamagnetic nanoparticle-ligand conjugate and then detecting the nanoparticle-ligand conjugate bound to the tissue. The subject is preferably a mammal susceptible to prostate cancer, such as a human or dog. More preferably, the subject is a human. Detection of conjugate bound to the tissue is indicative of prostate cancer, either primary or metastatic (depending on the observed location of the conjugate) or a precancerous condition in the subject. Preferably, the conjugate is found bound to cancerous prostate tissue at levels at least twice as high, and more preferably five times as high, as found bound to non-cancerous prostate tissue.

The paramagnetic or superparamagnetic nanoparticle-ligand conjugate that selectively binds to a component on the surface of a prostate cancer cell of the invention allows prostate cancer tissue to be detected using magnetic resonance imaging (MRI) or any other technique involving the application of an electrical, electromagnetic or magnetic field. For example, direct detection of the nanoparticles can be accomplished with highly sensitive superconducting quantum interference devices (SQUIDs). See Grossman et al., Proc Natl Acad Sci USA. 2004 Jan. 6; 101(1):129-34; Flynn et al., Phys Med Biol. 2005 Mar. 21; 50(6):1273-93.

MRI is a preferred method for detecting prostate cancer cells that have been bound by paramagnetic or superparamagnetic nanoparticle-ligand conjugates. The paramagnetic or superparamagnetic nanoparticle-ligand conjugate functions as a contrast agent when placed in a strong magnetic field by affecting the magnetic properties of the protons (hydrogen nuclei) contained in and surrounding the prostate cancer tissue to which the superparamagnetic nanoparticle-ligand conjugate is bound. MRI uses one or more very powerful magnets (e.g., magnets generating 5,000 to 20,000 gauss) to form a homogenous magnetic field. The MRI scanner then applies a radiofrequency pulse specific for hydrogen that causes the protons affected to precess in a different direction. Three gradient magnetic fields are then rapidly turned on and off to alter the main magnetic field at a very local level. When the RF pulse is turned off, the hydrogen protons return to their natural alignment within the magnetic field and release excess stored energy, giving a signal that is picked up by a coil and evaluated by Fourier transformation to create an image for viewing.

Contrast agents are generally used to enhance the image created by MRI. One advantage of the paramagnetic or superparamagnetic nanoparticle-ligand conjugates of the present invention is that they may be specifically targeted to prostate cancer tissue, allowing more precise image enhancement and delivery of smaller amounts of contrast agent. The paramagnetic or superparamagnetic nanoparticles also provide superior image quality. In evaluating magnetic materials as MR contrast agents, the ability of materials to shorten proton relaxation time can be more important than bulk magnetic properties such as magnetization. Since MR imaging works by determining the rates of two types of proton relaxations in various tissues and, by using variations in those relaxation rates to develop an image, the differences in proton relaxation times between the tissues must be sufficiently great to obtain a good quality image. MR contrast agents work by shortening proton relaxation time, thus increasing the contrast and overall image quality. Two relaxation parameters, termed spin-lattice relaxation time (T₁) and spin-spin relaxation time (T₂) are used in the generation of the MR image. The high relaxivity of the materials of the invention increase their effectiveness as MR contrast agents because it results in large effects on the MR image from small doses of iron.

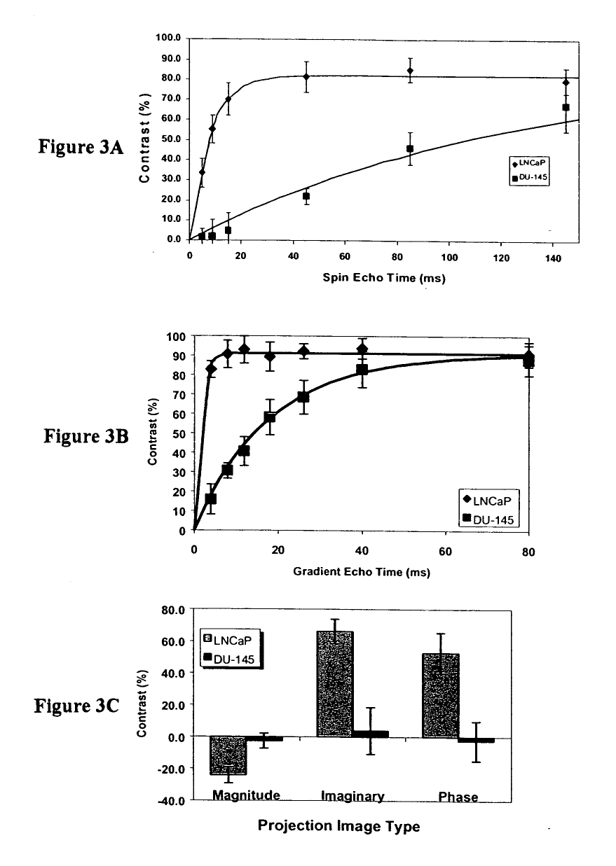

In further embodiments of the method of detecting prostate cancer cells, different types of MRI imaging techniques may be used. For example, spin-echo, gradient-echo, and FID projection images of samples evaluated by MRI may be used. In a preferred embodiment, gradient-echo imaging is used as it may provide greater contrast difference than that provided by other imaging techniques. Measured contrast values for various imaging techniques used in embodiments of the invention are provided, for example, by FIG. 3.

Methods of detecting prostate cancer of the present invention include contacting prostate tissue of the subject with the paramagnetic or superparamagnetic nanoparticle-ligand conjugate. In order to bring the nanoparticle-ligand conjugates into contact with the prostate tissue, the particles may be administered to the subject being studied. The nanoparticle-ligand conjugate can be administered to the subject in any convenient manner, including systemic administration (e.g., ingestion) or local administration (e.g., injection into or near the prostate). A route of administration that rapidly brings the nanoparticle-ligand conjugates into contact with prostate tissue is preferred. For example, the nanoparticle-ligand conjugates may be administered intravenously. Intravenous administration is preferred, is this will increase the likelihood of metastatic prostate cancer being detected. The nanoparticle-ligand conjugates may be formulated as known by those skilled in the art. For example, the nanoparticle-ligand conjugates may be delivered intravenously using a phosphate buffered saline solution. A variety of dosage levels are suitable for use with the invention. For example, a preferred dosage of iron oxide-containing nanoparticles for intravenous administration is between 1 mg and 5 mg of nanoparticle per 1 kg body weight of the subject. Alternatively, the nanoparticle-ligand conjugates may be brought into contact with prostate cancer tissue that has been obtained from the subject by, for example, a biopsy procedure.

Treatment of Prostate Cancer Cells Using Nanoparticle-Ligand Conjugates

The nanoparticle-ligand conjugate can also be used for treatment of prostate cancer or a precancerous condition. Thus, another aspect of the invention provides a method for treating prostate cancer in a subject that includes contacting prostate tumor tissue of the subject with the nanoparticle-ligand conjugate such that the conjugate binds to the tumor tissue; and irradiating the conjugate bound to the tumor tissue to result in thermal ablation of the tumor tissue. Irradiation may be accomplished using, for example, radiowaves (low frequency AM band and below) to heat the nanoparticles conjugates bound to the tumor cells to cause the destruction of the tumor cells by thermal ablation. Preferably, the radiowaves have a frequency between about 200 and 1,000 KHz. The nanoparticle conjugates can be delivered to the prostate tumor cells as described herein, for example by intravenous injection. Hilger et al. provide further description of the use of thermal ablation of tumors by magnetic nanoparticles (Hilger et al., Invest. Radiol. 2002 October; 37(10):580-586).

Treatment can be prophylactic or can be initiated upon or after the development of cancer. Treatment that is prophylactic, i.e., initiated before a subject manifests cancer symptoms, is referred to herein as treatment of a subject that is “at risk” of developing a disease. An example of a subject that is at risk of developing cancer is a person having a risk factor, such as a genetic marker, that is associated with the disease. Examples of genetic markers indicating a subject has a predisposition to develop prostate cancer include alterations in hereditary prostate cancer 1 (HPC1). Treatment initiated after the development of cancer may result in decreasing the severity of the symptoms of one of the conditions, or completely removing the symptoms.

Treatment by thermal ablation using nanoparticle-ligand conjugates may be used in conjunction with other methods such as, for instance, radiation therapy, hormonal therapy, surgery, cryosurgical ablation, the use of other antitumor agents, or a combination thereof. Other methods may be used before, after, or simultaneous to treatment by thermal ablation. A wide variety of antitumor agents are available that may be used (see, for example, Fischer et al., eds., The Cancer Chemotherapy Handbook, 6^{th} ed., (2003)). Antitumor agents that have proven particularly effective in treating prostate cancer include, for instance, cyclophosphamide, methotrexate, taxotere, doxorubicin, 5-fluorouracil, cisplatin, mitomycin C, and decarbazine.

The present invention is illustrated by the following examples. It is to be understood that the particular examples, materials, amounts, and procedures are to be interpreted broadly in accordance with the scope of the invention as set forth herein.

### EXAMPLES

### Example 1

### Cell Cultures and Evaluation of the Extent of PSMA Expression in Prostate Cell Lines

In order to determine the extent of PSMA expression in human tissue and cultured prostate cell lines, polymerase chain reaction and flow cytometry were performed. The expression of PSMA was examined by RT-PCR and flow cytometry in human tissues and in several cultured human prostate cancer cell lines. The findings indicate that it is highly-expressed in LNCaP and C4-2 cells, but almost absent in DU-145 and PC-3 cells. Monoclonal antibody MAb 3C6 conjugated to SPIONs selectively bound to LNCaP cells was readily detected as a significant change in signal intensity in magnetic resonance images obtained by standard methods. The same 3C6-conjugated SPIONs showed weak binding to DU-145 cells, giving rise to only small signal perturbations in the MR images.

Materials

Fluorescein-conjugated streptavidin was obtained from Molecular Probes (Eugene, Oreg.). Anti-PSMA antibody (clone 3C6) was purchased from Northwest Biotherapeutics (Bothell, Wash.). Antibody was biotinylated using EZ-LINK Sulfo-NHS-LC-Biotin from Pierce (Rockford, Ill.). The prostate cancer cell lines LNCaP, PC-3, and DU-145 were purchased from the American Tissue Type Collection (Manassas, Va.). The C4-2 prostate cancer cell line was a kind gift from Dr. G. N. Thalmann (University of Bern, Switzerland). PrEC cells were purchased from Cambrex, Inc. (East Rutherford, N.J.).

Cell Culture

LNCaP cells were cultured in RPMI 1640 medium (Sigma, St. Louis, Mo.) supplemented with 10% fetal calf serum (FCS); C4-2, PC-3, and DU-145 were cultured in T-medium with 10% FCS (Thalmann et al., Cancer Res 1994; 54:2577-2581); PrEC cells were cultured in PrEGM medium (Cambrex, East Rutherford, N.J.). All cells were cultured at 37° C. in a humidified 5% CO₂ atmosphere. Upon reaching 90% confluency, cells were collected either by detachment in 0.5% trypsin containing 0.02% EDTA or detached in cold phosphate buffered saline (PBS) using cell scrapers. Primary prostate cancer and normal prostate cells were isolated from a surgically resected prostate (University of New Mexico Hospital) and cultured in PrEGM medium (Cambrex) in accordance with policies of the University of New Mexico Human Research Review Committee.

Real-Time Reverse Transcriptase Polymerase Chain Reaction (RT-PCR)

Total RNA was isolated from cells using the RNeasy kit (Qiagen, Valencia, Calif.). Contaminating DNA was removed by DNase I treatment (Clontech, Palo Alto, Calif.). cDNA was synthesized using AMV RT (Promega, Madison, Wis.) and random hexamer primers (Amersham, Piscataway, N.J.) in the presence of RNase inhibitor (Promega). Real-time RT-PCR was performed in a 7000 Sequence Detection System thermocycler (Applied Biosystems, Foster City, Calif.) using gene-specific primer pairs and fluorescent reporter probes (Integrated DNA Technologies, Coralville, Iowa). To avoid amplification from genomic DNA, intron-spanning primers were used (designed with Primer Express software; Applied Biosystems). The primers and probe for PSMA were: 5′-tgagagactccaggactttgacaa-3′ (forward) (SEQ ID NO:1), 5′-ggatcaataaatgctctttccagaa-3′ (reverse) (SEQ ID NO:2), and 5′-agcaacccaatagtattaagaatgatgaatgatcaactca-3′ (probe) (SEQ ID NO:3). For glyceraldehyde-3-phosphate dehydrogenase (GAPDH), commercially available reagents (Applied Biosystems) were used. 350 ng of cDNA were used in a total volume of 25 μl of PCR MasterMix containing 900 nM of the primers and 300 nM of the probe. The cycling conditions were 95° C. 10 min, 45× (95° C. 15 sec, 60° C. 1 min). No-template and non-reverse transcribed RNA controls were included. The data was analyzed using the Sequence Detection System software (Applied Biosystems). The expression differences were calculated by the 2^{-ΔΔCt} method for assessing relative expression (Applied Biosystems, User Bulletin #2, 2001). The signals for PSMA were normalized to the signals for GAPDH to control for RNA input. The size of the amplicons was verified in agarose gels.

Flow Cytometry

PC-3 and DU-145 cells were cultured in RPMI 1640 (CELLGRO from Fisher, Pittsburgh Pa.) supplemented with 5% Fetal Bovine Serum (Hyclone, Logan, Utah). The other cell lines were cultured as described above. All cells were harvested by trypsinization, centrifuged and resuspended in PBS containing 0.1% azide and 1% BSA. Cells were analyzed for cell surface expression of PSMA using either biotinylated or unconjugated MAb 3C6. Fluorescent labeling was achieved with either a fluorescein conjugate of streptavidin or with a phycoerythrin conjugate of rat anti-mouse antibody (BD Biosciences, Palo Alto, Calif.). Data were acquired using a Becton Dickinson FACscan flow cytometer equipped with a 488-nm argon laser and CellQuest software (San Jose, Calif.).

Fluorescent Antibody Conjugation

Sulfo-NHS-LC-Biotin groups were attached to primary amines of MAb 3C6 using the EZ-LINK Sulfo-NHS-LC-Biotin kit according to the manufacturer's protocol. Conjugated MAb 3C6 was separated from low molecular weight compounds using a Millipore ULTRAFREE-MC Centrifugal Filter Unit at a force of 3000 gravities (g) in a fixed angle microfuge rotor. Antibody concentration was determined using the Pierce Protein Assay Reagent Kit, and the ratio of biotin/antibody was determined with a Pierce HABA colorimetric assay, both according to the manufacturer's protocol.

Expression of PSMA as Evaluated by Polymerase Chain Reaction

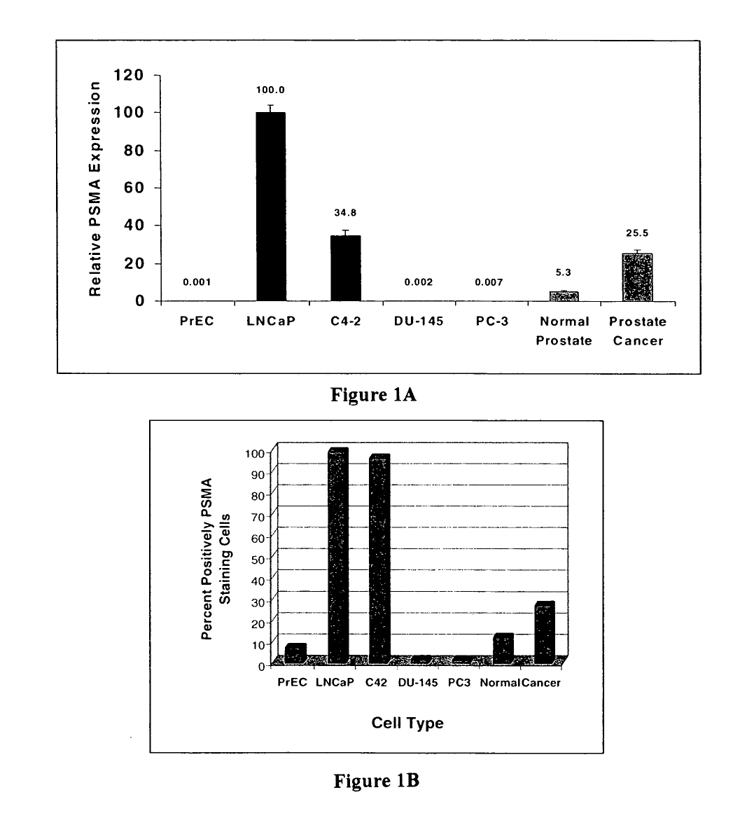

In order to determine the extent of PSMA expression in human tissues and cultured prostate cell lines, polymerase chain reaction assays were performed to measure the amount of PSMA messenger RNA produced. As shown in FIG. 1, real-time RT-PCR revealed significant expression of PSMA in both the LNCaP and C4-2 cell lines, while it was virtually undetectable in PrEC, DU-145 and PC-3 under the PCR conditions used. These results are in agreement with previously published studies (Denmeade et al., Prostate 2003; 54(4):249-57; Smith-Jones et al., J Nucl Med 2003; 44:610-617). PSMA expression in RNA derived from a prostate cancer specimen and its associated matched normal tissue was also measured. As previously reported (Yao et al., Semin Urol Oncol 2002; 20(3):211-8), PSMA expression was greater (by approximately 5-fold) in tumor as compared to normal tissue.

Flow Cytometry Evaluation of PSMA Expression

Since there is often an imperfect correlation between messenger RNA amounts in cells and the amounts of the translated proteins, flow cytometry was performed to further investigate the levels of cell surface expression of PSMA. The cultured cell lines were stained with MAb 3C6, followed by a PE (phycoerythrin)-conjugated secondary antibody, and examined by flow cytometry. The LNCaP and C4-2 cells expressed roughly 100-fold more PSMA on their surfaces (FIG. 1) than did either the DU-145 or PC3 cells. The primary normal and tumor cells and the PrECs expressed intermediate (7-27% PSMA-positive cells) amounts of cell-surface PSMA. The human cancer cells display a 2.3-fold greater amount of PSMA than do the normal cultured prostate cells. These results are in agreement with similar reports in the literature on these particular cell lines (Murphy et al., Urology 1998 May; 51(5A Suppl):89-97; Noss et al., Anticancer Res 2002 May-June; 22(3):1505-11). The fact that C4-2 cells retain PSMA expression indicates that they share the same phenotype as their LNCaP parent line. Based on these results, it was decided to further examine the LNCaP and DU-145 cells for differences in their ability to bind SPION-conjugated 3C6 antibodies.

### Example 2

### Detection of Bound SPION-Conjugated 3C6 Antibodies Using MRI

Using the cell lines described above, the ability to bind and detect PSMA on the cells using superparamagnetic nanoparticle-ligand conjugates was evaluated.

Antibody Conjugation to Superparamagnetic Beads and Cell Labeling

DYNABEADS MyOne Streptavidin was obtained from Dynal Biotech (Oslo, Norway), and MACS Streptavidin Microbeads were obtained from Miltenyi Biotec (Bergisch Gladbach, Germany). DYNABEADS MyOne Streptavidin superparamagnetic beads (1.05+/−0.10 μm diameter, 37% iron oxide w/w, polystyrene coating) and MACS Streptavidin MicroBeads (50 nm diameter, 55-59% iron oxide w/w, dextran coating) were used as contrast agents for MR imaging. DYNABEADS MyOne Streptavidin beads are superparamagnetic iron oxide particles with a polymer coating and an size of about 1 μm that are available pre-coated with streptavidin. MACS Streptavidin MicroBeads are superparamagnetic iron oxide particles with a dextran coating and a size of about 50 nm that are available precoated with streptavidin.

The DYNABEADS solution contained detergent (0.01% Tween 20) and preservative (0.09% sodium azide), which were removed by multiple washing with phosphate buffered saline (PBS). The MACS solution contains 0.05% sodium azide, but no detergent, and was not washed. LNCaP and DU-145 cell suspensions were incubated with biotinylated anti-PSMA antibody for 30 minutes at 4° C., followed by washing with PBS. Labeled cells were incubated with the streptavidin superparamagnetic beads (10 μl bead solution/10⁷ cells) for 30 minutes at 4° C. with gentle agitation. Cells were separated from unbound beads by repeated centrifugation at 300×g. LNCaP and DU-145 cells were each resuspended in 100 μl low melting point agarose and layered into a plastic tube as described below.

MRI Sample Preparation

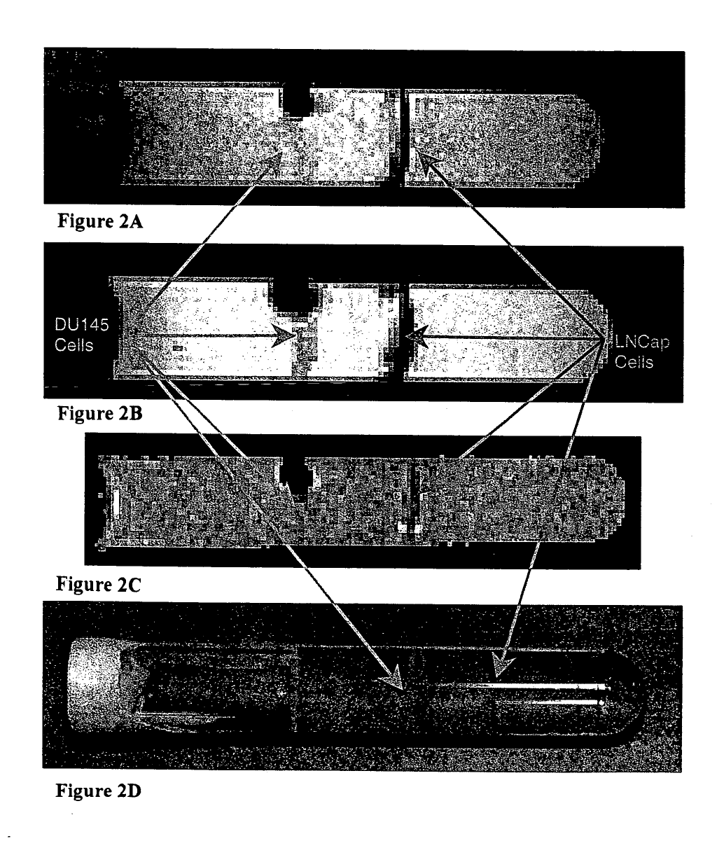

For the DYNABEADS sample, a layer of cell-free agarose gel was added to a 10 millimeter (mm) I.D. plastic tube, followed by a 2 mm layer of agarose containing 5×10⁶ superparamagnetic iron oxide nanoparticle (SPION)-labeled LNCaP cells, another layer of cell-free agarose, a 2 mm layer of agarose containing 5×10⁶ SPION-labeled DU-145 cells, and a final layer of cell-free agarose. The final structure of the DYNABEADS sample, which also contained an air bubble, is shown in FIG. 2D. The MACS sample was similarly prepared, except that the DU-145 cell layer was added first and there is no air bubble.

Magnetic Resonance Imaging

All MR imaging was carried out in a 1.9 T Oxford horizontal bore magnet using a Tecmag Libra NMR spectrometer, a Resonance Research shielded gradient set, and a Morris Instruments birdcage probe. The proton frequency was 80.35 MHz.

T₂-weighted images were acquired using a standard 2D spin echo (SE) imaging sequence (TR=1.1 second (s), NEX=8) with the echo time (TE) varying from 5.5 millisecond (ms) to 145.5 ms. The 2D spin echo sequence (with TE=5.5 ms) was also used to acquire T₁-weighted images by varying the repetition time (TR) from 0.5 s to 12 s. T₂*-weighted images were acquired using a standard 2D spoiled gradient-recalled echo (GRE) imaging sequence (TR=1.1 s, NEX=8) with TE varying from 4 ms to 80 ms. All 2D images were acquired with a 128×32 image matrix, 0.5 mm in-plane resolution, and a slice thickness of 2.5 mm.

The three-dimensional (3D) FID projection images were made with modifications to the first MRI technique (Lauterbur P C. Nature 1972; 242:190-191; Glover et al., J Magn. Reson Imaging 1992; 2:47-52; Kuethe et al., Magn Reson Med 1998; 39:85-88). A radio frequency pulse excites spins in the presence of the imaging gradient, and detection of the resulting signal (the free-induction decay or FID) begins immediately (TE=0). The sequence is T₁-weighted using a TR of 3.5 ms and a 9° excitation pulse. Two sets of data, one using a given gradient and one using its negative (opposite direction) are required to make a projection of the object in one direction. Projections in 26850 directions, roughly equally spaced in solid angle, were obtained, resulting in a total imaging time of 12.6 minutes. From these data, a 3D image was constructed using Fourier projection. The resulting image matrix is 140³, with isotropic 0.367 mm resolution. The time-domain data are collected in quadrature (i.e., as complex numbers), allowing reconstruction of images using the magnitude, phase, or only the real component or the imaginary component of the data.

Using a series of SE images with different TE values, T₂ decay curves were generated for each of the three DYNABEADS sample components by selecting a region of interest (ROI) corresponding to each component (LNCaP cells, DU-145 cells, and agarose gel) and plotting the mean signal intensity S of the ROI as a function of TE. T₂* decay curves were obtained from the GRE images using the same procedure. The time constants T₂ and T₂* were determined by fitting the decay curves with a decaying exponential, S(TE)=S(0)*exp(−TC/TE), where S(0) is the signal intensity for TE=0, and TC is the desired time constant.

For the MACS sample, T₁ recovery curves were generated using a series of one-dimensional SE images with TE=10.7 ms. (The MR signal was spatially resolved only along the axis of the test tube.) The SE imaging sequence was preceded by an inversion (π) pulse and a variable time interval, during which the sample magnetization was allowed to recover toward equilibrium. The recovery times ranged from 3 ms to 8 seconds (s). ROIs were selected corresponding to the LNCaP cells, the DU-145 cells, and the agarose gel. The signal intensity S of an ROI was plotted as a function of recovery time t, and T₁ was determined by a 3-parameter fit of the function S(t)=S(∞)*(1−P*exp(−T₁/t)), where S(∞) is the fully-recovered signal intensity and P is a measure of the level of inversion achieved (P=2 for perfect inversion).

Contrast (%) was defined as C=100(I_{C}/I_{A}−1), where I_{C} is the MR signal intensity from the pixels in the region of interest surrounding the labeled cells, and I_{A} is the MR signal intensity of the agarose pixels. Note that contrast as defined here can range from +100% to −100%, with positive contrast indicating that the object of interest is hyperintense with respect to the background. The average and standard deviation of the pixel intensities in the regions of interest were measured with MiraAP software (Axiom Research, Inc., Tucson, Ariz.). The errors in the computed contrast were calculated using a standard propagation-of-errors analysis (Bevington P R. Data Reduction and Error Analysis for the Physical Sciences. New York: McGraw-Hill; 1969. p. 56-65) based on the measured standard deviations of the pixel intensities for the cells (σ_{C}) and agarose (σ_{A}). The theoretical dependence of the contrast on time was derived as follows. For the T₂ and T₂* experiments with the DYNABEADS, the MRI signal intensity is given by I(t)=Iₒexp(−t/Tᵢ), where Tᵢ is the relaxation time for the ith sample component (LNCaP cells, DU-145 cells, or agarose gel). The time-dependence of the contrast between the cells and the agarose is then C(t)=100*{1−exp[−t*(1/T_{C}−1/T_{A})]}, where T_{C(A)} is the relaxation time for the cells (agarose). Hence, the contrast recovers exponentially with a time constant given by the difference between the relaxation rates for the cells and the agarose.

The treatment of the contrast for the MACS beads differs only in that these agents primarily altered T₁. The time-dependence of the contrast for the T₁-weighted spin echo experiment is C(t)=100*{1−[1−exp(−t/T_{1C})]/[1−exp(−t/T_{1A})]}, since the signal intensity of the ith component is proportional to [1−exp(−t/T₁ᵢ)] for this experiment.

MRI of DYNABEADS Sample

The MRI samples were prepared with layers of DYNABEADS SPION-labeled LNCaP and DU-145 cells, in between layers of agarose gel, in order to measure the MRI signal intensities of the control DU-145 cells, the PSMA-positive LNCaP cells, and the agarose simultaneously in the same sample. FIG. 2 shows three MRI images, obtained using spin echo (A), gradient echo (B) and FID projection imaging (C), and a photograph of the DYNABEADS sample. In all three MRI images, the LNCaP cells appear hypointense against the agarose.

The image contrast for spin-echo, gradient-echo, and FID projection images of the DYNABEADS sample is given in FIG. 3. A series of spin-echo images of the DYNABEADS sample were taken at echo times (TE) ranging from 5.5-145.5 ms in order to study the T₂ contrast. For the spin-echo sequence, the contrast difference between the PSMA-expressing LNCaP cells and the poorly-expressing DU-145 cells was maximal at an echo time of 15.5 ms (FIG. 3A). It was expected that the contrast between the labeled cells and the background could be improved using a gradient-echo imaging sequence, which doesn't refocus dephasing due to static field gradients, unlike the spin-echo sequence. For example, the size of the serendipitous air bubble is larger in the gradient-echo image (FIG. 2B) than it is in the spin-echo image (FIG. 2A) due to this effect. In general, the contrast difference for a given TE is better in the gradient-echo images (FIG. 3B) than in the spin-echo images. For the gradient-echo sequence, the best contrast between the two cell types is obtained for the image with TE≦4 ms. Thus, using a TE shorter than 4 ms may further improve contrast because the echo time-dependence of the contrast for the LNCaP cells is approximately linear for the shortest echo times.

FIG. 2C shows one plane of the imaginary component of a 3D FID projection image of the DYNABEADS sample. Because the effective TE is zero for FID projection imaging, there is no signal loss in the magnitude image (not shown) due to the superparamagnetic particles. The imaging procedure is instead sensitive to the shift in NMR frequency that accompanies the higher magnetic field strength in the vicinity of the particles. Thus, the magnitude image shows only a minor displacement of the signal away from its true location; however, the imaginary component of the image (FIG. 2C) shows the phase shift caused by the higher spin frequency, resulting in very good contrast for detecting the nanoparticles. Only the LNCaP cells show up well in the FID projection image, although there is a barely-perceptible shift in the magnitude image at the position of the DU-145 cells. The DU-145 cells are not detectable in either the imaginary or phase images. In the FID projection image, the best contrast is observed in the imaginary component of the complex image (FIG. 3C).

In the DYNABEADS sample, the longitudinal relaxation time T₁ for the water in all of the regions of the sample (agarose, DU-145 cells, and LNCaP cells) was found to be roughly 1.6 seconds by determining the recovery time that nulled the signal using a standard inversion-recovery pulse sequence. The relative signal intensities of the three sample components were found to be independent of the repetition time TR for a series (not shown) of spin-echo images (TE=5.5 ms, TR=0.2, 1.0, 3.0 s) demonstrating that the water protons surrounding all three sample components relaxed with the same T₁. Therefore, the observed contrast in the MRI of the DYNABEADS sample arises solely from transverse relaxation (T₂) and dephasing (T₂*) effects. Transverse relaxation times T₂ were determined for each of the sample components by exponential fitting of the mean pixel intensity in spin-echo images vs. echo time as described in the methods section. The results are summarized in Table 1, below, which shows the NMR relaxation parameters of MACS® and Dynabeads® samples. The relaxation time T₂* was determined for the DU-145 cells by exponential fitting of the mean pixel intensity in gradient-echo images vs. echo time, resulting in T₂*=20.4±0.8 ms. The decay of the agarose signal intensity could not be fit to either an exponential or Gaussian function, due to the inhomogeneity of the applied magnetic field; defining T₂* to be the time at which the signal is down by a factor of e results in an estimate of T₂* greater than 100 ms. T₂* for the majority of the LNCaP region is so short that the NMR signal has already decayed by ˜7-fold at the time of the first echo measurement (4 ms), implying T₂*≦2 ms. The measured values of T₂ and T₂* for each sample component were used to calculate the contrast vs. time curves (solid lines) in FIG. 3.

TABLE 1

| | MACS ® | | Dynabeads ® | | |
|---|---|---|---|---|---|
| | T₁ (s) | r₁C (s⁻¹) | T₂* (ms) | T₂ (ms) | r₂C (s⁻¹) |
| agarose gel | 2.25 ± 0.08 | — | ˜100 | 98 ± 1 | — |
| LNCaP | 1.48 ± 0.05 | 0.23 | ˜2 | 9 ± 1 | 100 |
| DU-145 | 1.87 ± 0.05 | 0.090 | 20 ± 1 | 56 ± 1 | 7.7 |

MRI of MACS Sample

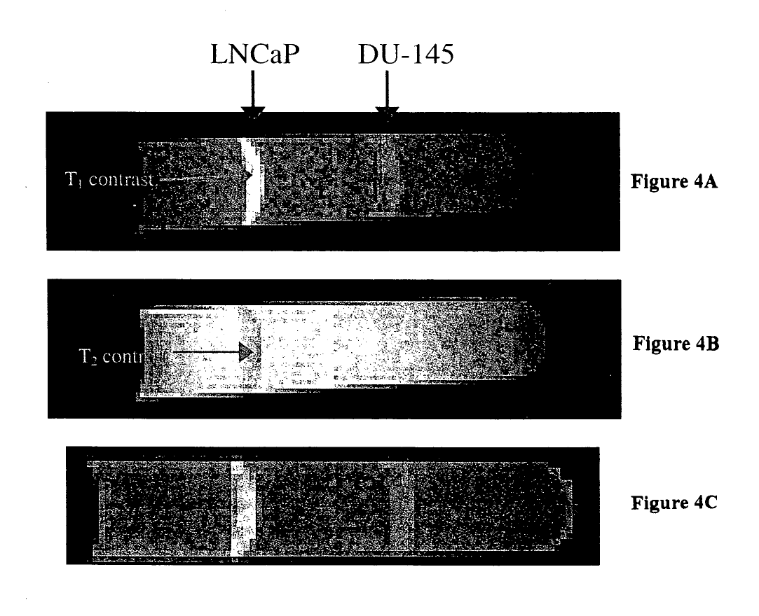

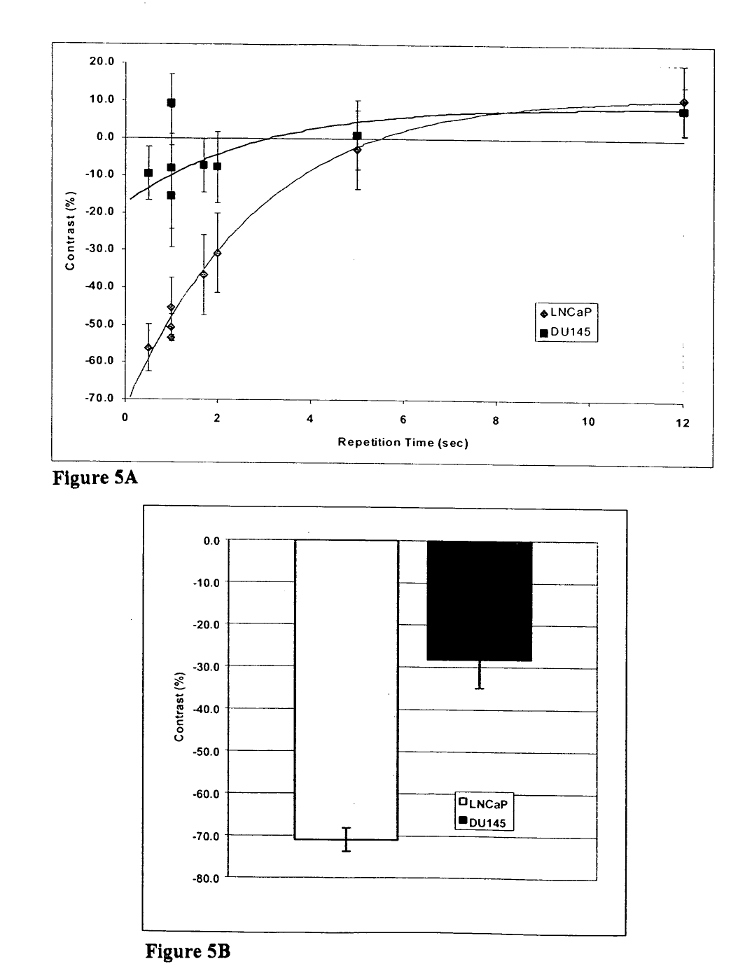

The MACS Microbeads resulted in very different MR image contrast from that induced by the DYNABEADS. In this case, the SPION-labeled cells appear brighter relative to the background agarose gel, due to the shortening of the longitudinal relaxation time T₁ of water in the vicinity of magnetic particles (FIGS. 4A and 4C). Weak T₂ (dark) contrast of the LNCaP cells is apparent in the spin echo image acquired with TR=12 s (FIG. 4B), in which the LNCaP cells appear with slightly lower signal intensity than the background agarose gel. A TR of 12 s is sufficiently long that the magnetization of the entire sample is fully recovered, eliminating the T₁ contrast and allowing the weaker T₂ contrast to be observed. Gradient-echo images of the MACS sample (not shown) with similar TR values showed contrast very similar to the spin-echo images. The T₁-weighted FID projection magnitude image (FIG. 4C) is very similar to the spin-echo image with TR=1 s.

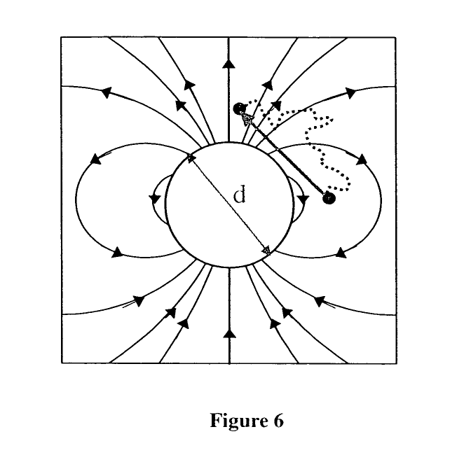

The contrast as a function of repetition time (TR) for spin echo images of the MACS sample is given in FIG. 5A. At shorter values of TR, the contrast in the spin-echo images (FIG. 5A) is much better for the LNCaP cells than for the DU-145 cells. The contrast achieved in the FID projection magnitude image of the MACS sample (FIG. 5B) was also much better for the LNCaP cells than for the DU-145 cells. T₁ was determined for each sample component from a series of 1-dimensional inversion-recovery prepared SE images (TE=10.7 ms). All three sample components showed single exponential recoveries resulting in the T₁ values given in Table 1. The smooth curves through the data in FIG. 5 were calculated using the measured values of T₁ for each sample component.

Differences in MRI Contrast from DYNABEADS and MACS SPIONs

Generally, superparamagnetic nanoparticles cause dark contrast using standard MR imaging methods (Wang et al., Eur Radiol 2001; 11(11):2319-31; Anzai Y., Top Magn Reson Imaging 2004; 15(2):103-11). The MRI signal is decreased in the vicinity of the magnetic particles because the transverse relaxation rate (1/T₂) is increased. Transverse relaxation is the loss of phase coherence of the proton nuclear spin signal, here caused by water protons diffusing in the magnetic field gradients from the SPIONs. Compared to the DYNABEADS, the MACS beads would be expected to cause weaker T₂/T₂* effects, as observed here, because each MACS bead contains much less iron oxide and therefore has a much smaller magnetic moment.

However, the MACS beads do cause a significant enhancement of the longitudinal relaxation rate (1/T₁), which dominates the image contrast for most choices of imaging parameters. In considering T₁ contrast, the salient difference between the DYNABEADS and MACS beads is their size: the DYNABEADS are 20 times larger in diameter than the MACS beads. As a result of their smaller size, the magnetic field due to the MACS beads varies more rapidly as a function of position than field due to the larger DYNABEADS. This has important consequences for T₁, as illustrated in FIG. 6. Longitudinal relaxation is caused by fluctuations in the magnitude and direction of the magnetic field experienced by each nuclear spin. The timescale of the fluctuations is important, because the longitudinal relaxation rate is a maximum when the correlation time, υ_{c}, characterizing the fluctuations is roughly equal to the inverse of the nuclear Larmor frequency, 1/ωₒ (Slichter C P. Principles of Magnetic Resonance. Springer-Verlag, Berlin, 1990) (˜2 ns in this example). To compare the timescale of the magnetic field fluctuations for water in the vicinity of the MACS beads and DYNABEADS, the diffusion constant, D, of water at 25° C. is 2.2×10⁻⁹ m²/s, and that the root-mean-square displacement for diffusion in three dimensions is √(6Dt), where t is the elapsed time, was considered. In order for a water molecule diffusing near a MACS bead to travel from a region where the magnetic field of the particle points in one direction to a region where the magnetic field is pointing in the opposite direction, the molecule must diffuse roughly 50 nm (the diameter of the bead), which requires 190 ns. For the DYNABEADS, the water molecule must diffuse 20 times farther in order to experience a reversal of the field direction, which requires 76 μs (400 times longer). The correlation time for the MACS beads is therefore considerably closer to 1/ωₒ, resulting in a relaxation rate, 1/T₁, closer to the maximum value.

In addition to producing different types of contrast, DYNABEADS and MACS streptavidin beads, conjugated to MAb 3C6, show a different degree of specificity in their in vitro binding to LNCaP and DU-145 cells. To determine the relative concentration of SPIONs bound to the LNCaP and DU-145 cells, the relaxation rate (1/relaxation time) in the presence of a contrast agent is given by Rₙ=Rₙₒ+rₙC, where Rₙₒ is the relaxation rate in the absence of the contrast agent, r is the relaxivity (in units of s⁻¹mM⁻¹), C is the concentration (in millimoles (mM)), and n is 1 or 2 (for T₁ or T₂ relaxation), was considered. In Table 1, the products r₁C and r₂C have been determined by taking the difference between Rₙ, the relaxation rate of the SPION-labeled cells embedded in agarose gel, and Rₙₒ, the relaxation rate of neat agarose gel. For the DYNABEADS sample, the ratio of the r₂C products (LNCaP/DU-145) cells is 13, indicating that the concentration of SPIONs is 13 times higher in the LNCaP cell band than in the DU-145 cell band. For the MACS sample, the ratio of r₁C products indicates that the concentration of SPIONs is 2.6 times higher in the LNCaP band compared to the DU-145 band. For the DYNABEADS, the in vitro binding specificity derived from the measured T₂ values is excellent, as expected from the RT-PCR and flow cytometry results. Although the in vitro binding specificity of the antibody-conjugated MACS beads is poorer, they still produces a significant difference in T₁ contrast for the two cell types. According to the manufacturer, MACS beads will bind non-specifically to non-viable cells, which may explain their lower specificity. The difference in particle size may also affect how well unbound SPIONs centrifuge out of the cell mixture.

Summary and Conclusions

Although great progress has been made in the last decade in the development of physical imaging methods for cancer, there is still significant need for increased specificity, particularly for metastatic lesions. The increased expression of the cell surface antigen, PSMA, was exploited to target prostate tumor cells that differentially display this marker. Targeted MRI contrast agents were prepared by conjugating anti-PSMA monoclonal antibody 3C6 to commercial streptavidin SPIONs and have demonstrated the ability of these agents to specifically enhance the MRI image contrast of PSMA-expressing cancer cells in vitro. It was further demonstrated that these agents produce good contrast, both bright and dark, for a wide variety of MRI sequences, allowing for both specificity of detection and flexibility in the choice of imaging technique.

### Example 3

### In Vivo Detection of Tumor Tissue Using MRI

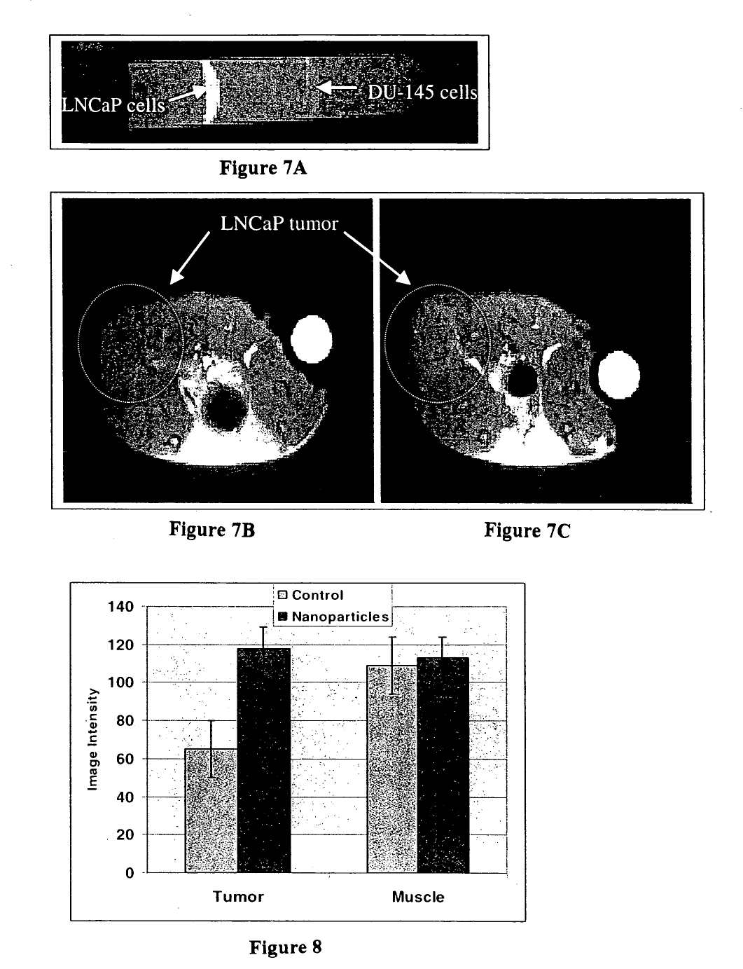

Nude athymic mice were obtained (Harlan, I N) and human LNCaP tumor cells were injected into the flank of the mouse where they developed into a large subcutaneous tumor. The mouse shown in FIGS. 7B and 7C has a large subcutaneous LNCaP tumor on its left flank. (The bright disk on the right side of these images is a CuSO₄-doped water standard.) MACS Streptavidin Microbeads were labeled with biotinylated anti-PSMA antibodies as described in Example 2 and administered by tail vein injection. FIG. 7A shows a T₁-weighted image (repetition time=0.5 seconds) of a test tube containing LNCaP cells and DU-145 (control) cells that were exposed to the same contrast agent. The T₁-weighted MR images of the mouse were also acquired with a repetition time of 0.5 seconds. In the image shown in FIG. 7B, which was acquired about 30 minutes after injection of the contrast agent, the tumor intensity is similar to that of muscle. At 23 hours post-injection (FIG. 7C), the tumor appears bright compared to the surrounding muscle, indicating that binding of the contrast agent to the tumor cells has occurred. Although the images presented in FIGS. 7B and 7C show the qualitative brightening of the tumor in the presence of PSMA-conjugated nanoparticles, measurement of the pixel intensities in the two images shown in FIG. 8 reveals just how robust this response was. The tumor brightened from 65±15 to 118±15, almost a factor of two in intensity, while the musculature brightness remained unchanged at 111-113±12 units (mean±standard deviation for 5,000-10,000 pixels).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. Any inconsistency between the material incorporated by reference and the material set for in the specification as originally filed shall be resolved in favor of the specification as originally filed. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### SEQUENCE LISTING FREE TEXT

SEQ ID NO:1: forward PSMA primer

SEQ ID NO:2: reverse PSMA primer

SEQ ID NO:3: PSMA probe

What is claimed is:

1. A nanoparticle-ligand conjugate comprising at least one paramagnetic or superparamagnetic nanoparticle; and at least one recognition ligand that selectively binds to a component on the surface of a prostate cancer cell.

2. The nanoparticle-ligand conjugate of claim 1 wherein the nanoparticle comprises iron oxide.

3. The nanoparticle-ligand conjugate of claim 1 comprising a plurality of recognition ligands.

4. The nanoparticle-ligand conjugate of claim 1 comprising a plurality of different recognition ligands.

5. The nanoparticle-ligand conjugate of claim 1 comprising a plurality of nanoparticles.

6. The nanoparticle-ligand conjugate of claim 5 further comprising a plurality of recognition ligands.

7. The nanoparticle-ligand conjugate of claim 5 further comprising a plurality of different recognition ligands

8. The nanoparticle-ligand conjugate of claim 1 wherein the recognition ligand binds to prostate surface membrane antigen (PSMA).

9. The nanoparticle-ligand conjugate of claim 1 wherein the recognition ligand comprises an antibody.

10. The nanoparticle-ligand conjugate of claim 6 wherein the antibody comprises a monoclonal antibody.

11. The nanoparticle-ligand conjugate of claim 1 wherein the nanoparticle comprises a superparamagnetic particle.

12. A method for diagnosing prostate cancer in a subject comprising:
contacting prostate tissue of a subject with the nanoparticle-ligand conjugate of claim 1;
applying a magnetic field to the prostate tissue; and
detecting nanoparticle-ligand conjugate bound to the prostate tissue;
wherein the presence of conjugate bound to the prostate tissue is indicative of prostate cancer in the subject.

13. The method of claim 12 wherein the recognition ligand binds to prostate surface membrane antigen (PSMA).

14. The method of claim 12 wherein the recognition ligand comprises an antibody.

15. The method of claim 12 wherein contacting prostate tissue of the subject with the nanoparticle-ligand conjugate is performed in vivo.

16. The method of claim 12 wherein contacting prostate tissue of the subject with the nanoparticle-ligand conjugate is performed in vitro.

17. The method of claim 12 wherein the nanoparticle-ligand conjugate is detected using molecular resonance imaging (MRI).

18. The method of claim 12 wherein the prostate tissue comprises metastatic prostate cancer tissue.

19. A method for treating prostate cancer in a subject comprising:
contacting prostate tumor tissue of the subject with the nanoparticle-ligand conjugate of claim 1 such that the conjugate binds to the tumor tissue; and
irradiating the tumor tissue to result in thermal ablation of the tumor tissue.

20. The method of claim 19 wherein the recognition ligand binds to prostate surface membrane antigen (PSMA).

21. The method of claim 19 wherein the recognition ligand comprises an antibody.

22. The method of claim 19 wherein the prostate tumor tissue comprises metastatic prostate cancer tissue.
